SEQUENCE LISTING (18 sequences)

SEQ ID NO: 1 - protein, 6 aa, Artificial Sequence
  Synthetic Polypeptide
HSSKLQ

SEQ ID NO: 2 - protein, 7 aa, Artificial Sequence
  Synthetic Polypeptide
KGISSQY

SEQ ID NO: 3 - protein, 7 aa, Artificial Sequence
  Synthetic Polypeptide
SRKSQQY

SEQ ID NO: 4 - protein, 7 aa, Artificial Sequence
  Synthetic Polypeptide
GQKGQHY

SEQ ID NO: 5 - protein, 7 aa, Artificial Sequence
  Synthetic Polypeptide
EHSSKLQ

SEQ ID NO: 6 - protein, 7 aa, Artificial Sequence
  Synthetic Polypeptide
QNKISYQ

SEQ ID NO: 7 - protein, 7 aa, Artificial Sequence
  Synthetic Polypeptide
ENKISYQ

SEQ ID NO: 8 - protein, 7 aa, Artificial Sequence
  Synthetic Polypeptide
ATKSKQH

SEQ ID NO: 9 - protein, 7 aa, Artificial Sequence
  Synthetic Polypeptide
KGLSSQC

SEQ ID NO: 10 - protein, 7 aa, Artificial Sequence
  Synthetic Polypeptide
LGGSQQL

SEQ ID NO: 11 - protein, 7 aa, Artificial Sequence
  Synthetic Polypeptide
QNKGHYQ

SEQ ID NO: 12 - protein, 7 aa, Artificial Sequence
  Synthetic Polypeptide
TEERQLH

SEQ ID NO: 13 - protein, 7 aa, Artificial Sequence
  Synthetic Polypeptide
GSFSIQH

SEQ ID NO: 14 - protein, 6 aa, Artificial Sequence
  Synthetic Polypeptide
HSSKXQ

SEQ ID NO: 15 - protein, 4 aa, Artificial Sequence
  Synthetic Polypeptide
SKLQ

SEQ ID NO: 16 - protein, 6 aa, Artificial Sequence
  Synthetic Polypeptide
HSSKLQ

SEQ ID NO: 17 - protein, 6 aa, Artificial Sequence
  Synthetic Polypeptide
HSSKXQ

SEQ ID NO: 18 - protein, 6 aa, Artificial Sequence
  Synthetic Polypeptide
HSSKLQ

(19) United States
(12) Patent Application Publication
(10) Pub. No.: US 2016/0097082 A1
(43) Pub. Date: Apr. 7, 2016

(54) PROSTATE SPECIFIC ANTIGEN PROTEOLYTIC ACTIVITY FOR CLINICAL USE

(71) Applicant: OHMX Corporation, Evanston, IL (US)

(72) Inventor: Dimitra Georganopoulou, Lincolnwood, IL (US)

(73) Assignee: OHMX Corporation, Evanston, IL (US)

(21) Appl. No.: 14/863,397

(22) Filed: Sep. 23, 2015

Related U.S. Application Data
(60) Provisional application No. 62/054,369, filed on Sep. 23, 2014.

(51) Int. Cl. C12Q 1/37; C12Q 1/68

(52) U.S. Cl. CPC C12Q 1/37; C12Q 1/6886; G01N 2333/96433; C12Q 2600/118; C12Q 2600/158; G01N 2800/7028; G01N 2800/52; G06F 19/26

(57) ABSTRACT

Methods for predicting the outcome of and monitoring prostate cancer patients using prostate specific antigen (PSA) proteolytic activity (PPA) in combination with other molecular biomarkers or other parameters are described. Methods of determining sets of parameters for use in predicting the outcome of and monitoring of prostate cancer patients are also described.

### RELATED APPLICATIONS

This application claims priority under 35 U.S.C. §119(e) to U.S. provisional patent application Ser. No. 62/054,369, filed Sep. 23, 2014, entitled “PSA PROTEOLYTIC ACTIVITY,” the contents of which are incorporated herein by reference in their entirety.

### FIELD OF THE INVENTION

Methods for predicting the outcome of and monitoring prostate cancer patients using prostate specific antigen (PSA) proteolytic activity (PPA) in combination with other molecular biomarkers or parameters are described. Methods of determining sets of parameters for use in predicting the outcome of and monitoring of prostate cancer patients are also described.

### BACKGROUND OF THE INVENTION

One in six American men will develop prostate cancer (PCa) in their lifetimes, making it the second leading cause of cancer death among males in North America. Although greater screening rates have drastically decreased mortality, they have also caused a controversial upsurge in overdiagnosis and overtreatment. Physicians and patients often choose clinical interventions even for non-aggressive prostate cancer, despite evidence that intervention may not be warranted. This intervention, however, places immediate undue physical, emotional, and financial burdens on the patients, which has led to the latest clinical guidelines recommending the reduction of screening altogether. Recent recommendations from the U.S. Preventative Services Task Force (USPSTF) and the American Urology Association (AUA) to discontinue routine PSA screening have helped to further fuel the controversy. These recommendations have created concern about the potential increase in mortality from “missed” cancers. There is a need for a test that addresses the root of the twin problems of overdiagnosis and overtreatment, while maintaining vigilant screening.

### SUMMARY OF THE INVENTION

Methods for using prostate specific antigen (PSA) proteolytic activity (PPA) alone or in conjunction with other parameters, such as the gene expression level of one or more molecular biomarkers, for predicting the outcome of prostate cancer in a subject, such as a patient diagnosed with prostate cancer, are described. Also described are methods of determining a set of parameters to predict the outcome of prostate cancer in a subject, such as a patient diagnosed with prostate cancer. The subject matter of the present invention involves, in some cases, interrelated products, and/or alternative solutions to a particular problem.

In some embodiments of any one of the methods provided herein, multiple variates, including, but not limited to, baseline clinical parameters and molecular biomarkers are analyzed in concert with PPA to take advantage of the synergistic effects of PPA and other biomarkers to enhance determinations of aggressiveness and/or prognosis, predictions of upstaging or biological recurrence, and monitoring of therapy and/or disease progression.

In some embodiments of any one of the methods provided herein, the subject is one already diagnosed with prostate cancer. In some embodiments of any one of the methods provided herein, the subject is also being diagnosed with prostate cancer at the time these methods are carried out.

In one aspect, a method for predicting outcome of prostate cancer in a subject, such as a patient diagnosed with prostate cancer, comprising a) determining or obtaining a level of prostate specific antigen (PSA) proteolytic activity (PPA) in a sample from the subject; b) determining or obtaining a gene expression level of one or more molecular biomarkers in the sample or another sample from the subject; c) performing a multi-variate analysis with the level of PPA and gene expression level of one or more molecular biomarkers and, optionally, one or more additional parameters of the subject; and d) comparing a result of the multi-variate analysis to a value, such as a cutoff value, to predict the outcome of prostate cancer in the subject is provided.

In another aspect, a method of determining a set of parameters to predict the outcome of prostate cancer in a subject, such as a patient diagnosed with prostate cancer, comprising a) determining or obtaining a level of PPA in a sample from a subject; b) determining or obtaining a gene expression level of one or more molecular biomarkers in the sample or another sample from the subject; c) performing one or more multi-variate analyses with the level of PPA and gene expression level of one or more molecular biomarkers and, optionally, one or more additional parameters of the subject; and d) determining the predictive power of the one or more multi-variate analyses is provided.

In some embodiments of any one of the methods provided herein, the predictive power is determined by comparing the results of the one or more multi-variate analyses to one or more values, such as one or more baseline, control, threshold or cutoff values. In some embodiments of any one of the methods provided herein, the predictive power is determined by calculating the area under the curve (AUC).

In some embodiments of any one of the methods provided herein, the level of PPA is normalized and it is the normalized level that is used in the multi-variate analysis. In some embodiments of any one of the methods provided herein, a logarithm of the level of PPA or normalized level of PPA is determined, and it is the logarithmic level that is used in the multi-variate analysis. In some embodiments of any one of the methods provided herein, the gene expression level of one or more molecular biomarkers is/are normalized and it is the normalized level(s) that is used in the multi-variate analysis. In some embodiments of any one of the methods provided herein, a logarithm of the gene expression level of one or more molecular biomarkers or normalized level(s) thereof is determined, and it is the logarithmic level(s) that is/are used in the multi-variate analysis. In some embodiments of any one of the methods provided herein the normalized level(s) is/are normalized with a normalization factor.

In some embodiments of any one of the methods provided herein, a step of determining a normalized level of PPA comprises contacting the sample with a labeled prostate cancer-specific peptide that is a substrate of PSA and normalizing the level of PPA with a normalization factor.

In some embodiments of any one of the methods provided herein, the normalization factors for normalizing the level of PPA and normalizing the gene expression level(s) are the same. In some embodiments of any one of the methods provided herein, the normalization factors are different.

In any one of the methods provided herein, the sample and/or other sample is a/an expressed prostatic secretion (EPS), post prostatic massage urine (PMU), PMU pellet, urine, semen, or prostatic fluid (PF) sample.

In some embodiments of any one of the methods provided herein, the normalization factor is serum PSA (sPSA), log of the serum PSA (sPSA), EPS sample volume, log of the EPS sample volume, PSA RNA copy number, log of PSA RNA copy number, amount of RNA, or log of amount of RNA. In some embodiments of any one of the methods provided herein, any one of the foregoing is a level of the factor in the same sample or a different sample from the subject.

In some embodiments of any one of the methods provided herein, the one or more molecular biomarkers comprise any one or more of GAPDH, PSA, TXNRD1, TXNRD2, TMPRSS2:ERG and PCA3 by quantitative reverse transcription polymerase chain reaction (qRT-PCR). In some embodiments of any one of the methods provided herein, the one or more molecular biomarkers comprises TXNRD2 or TMPRSS2:ERG or both TXNRD2 and TMPRSS2:ERG.

In some embodiments of any one of the methods provided herein, the additional parameters include, but are not limited to, age, Gleason score, a level of serum PSA, EPS volume, and prostate volume. In some embodiments of any one of the methods provided herein, the additional parameters include any one or more of (or any combination of) the additional parameters found in Tables 1-4. In some embodiments of any one of the methods provided herein the additional parameters are the additional parameters as found in any one of Tables 1-4.

In some embodiments of any one of the methods provided herein, the method further comprises i) creating a ROC curve based on one or more values from the multi-variate analysis; ii) identifying the point on the ROC curve closest to the coordinates (0,1); and iii) using the closest point to establish the cutoff value.

In some embodiments of any one of the methods provided herein, the method further comprises i) creating one or more ROC curves based on one or more values from the one or more multi-variate analyses; ii) identifying the point(s) on the one or more ROC curves closest to the coordinates (0,1); and iii) using the closest point(s) to establish one or more cutoff values.

In some embodiments of any one of the methods provided herein, a result of the multi-variate analysis is compared to a baseline, control, cutoff or a threshold level. In some embodiments of any one of the methods provided herein, when the result is less, poorer prognosis of the prostate cancer is indicated, while when the result is greater, better prognosis of the prostate cancer is indicated.

In some embodiments of any one of the methods provided herein, when a result of the multi-variate analysis is less, progression to a worse stage of the prostate cancer is indicated, while when the result is greater, no progression of the prostate cancer is indicated.

In some embodiments of any one of the methods provided herein, when the result is less, more aggressive prostate cancer is indicated, while when the result is greater, less or non-aggressive prostate cancer is indicated.

In some embodiments of any one of the methods provided herein, when the result is less, an upstaging or biochemical recurrence of the prostate cancer is indicated, while when the result is greater, an upstaging or biochemical recurrence is not indicated.

In some embodiments of any one of the methods provided herein, the method further comprises a step of treating the subject or providing information regarding a treatment to the subject based on information obtained from the multi-variate analysis. In some embodiments of any one of the methods provided herein, the treatment comprises clinical intervention. In some embodiments of any one of the methods provided herein, the method further comprises providing a different treatment to the subject based on the result(s) of the multi-variate analysis.

In some embodiments of any one of the methods provided herein, the method comprises further monitoring or recommending further monitoring of the subject without clinical intervention based on the result(s) of the multi-variate analysis. In some embodiments of any one of the methods provided herein, the monitoring includes the steps of any one of the methods provided herein repeated one or more additional times on one or more samples from the subject to monitor the prostate cancer in the subject.

In one aspect, a method for prognosis of prostate cancer in a subject, such as a patient diagnosed with prostate cancer, is provided. In one embodiment, said method comprises a) contacting a sample taken from said subject with a labeled prostate cancer specific peptide that is a substrate of prostate specific antigen (PSA), b) determining the level of PSA proteolytic activity (PPA) in said sample; c) normalizing said level of PPA to (i) total PSA in the serum of said subject (sPSA), (ii) said sample volume, (iii) combination of sPSA and said sample volume, and (iv) log transformed counterparts of any of i through iii; d) utilizing said normalized level of PPA to determine the prognosis of said prostate cancer in said subject. In an embodiment of such a method, one or more additional molecular biomarkers in said sample and, optionally, one or more clinical parameters of the subject are analyzed and used in conjunction with normalized level of PPA to determine the prognosis of prostate cancer in the subject.

In another aspect, a method for monitoring the progression of prostate cancer therapy and/or disease progression in a subject, such as a patient diagnosed with prostate cancer, is provided. In one embodiment, said method comprises a) contacting a sample taken from said subject with a labeled prostate cancer specific peptide that is a substrate of PSA, b) determining the level of PPA in said sample; c) normalizing said level of PPA to (i) sPSA of said subject, (ii) said sample volume, (iii) combination of sPSA and said sample volume, and (iv) log transformed counterparts of any of i through iii; d) utilizing said normalized level of PPA to monitor the progression of prostate cancer therapy and/or disease progression in said subject diagnosed with prostate cancer. In an embodiment of such a method, one or more additional molecular biomarkers in said sample and, optionally, one or more clinical parameters of the subject are analyzed and used in conjunction with normalized level of PPA to monitor the progression of prostate cancer therapy in the subject.

In another aspect, a method for determining aggressiveness of prostate cancer in a subject, such as a patient diagnosed with prostate cancer, is provided. In one embodiment, said method comprises a) contacting a sample taken from said subject with a labeled prostate cancer specific peptide that is a substrate of PSA, b) determining the level of PPA in said sample; c) normalizing said level of PPA to (i) sPSA of said subject, (ii) said sample volume, (iii) combination of sPSA and said sample volume, and (iv) log transformed counterparts of any of i through iii; d) utilizing said normalized level of PPA to determine the aggressiveness of said prostate cancer in said subject. In an embodiment of such a method, one or more additional molecular biomarkers in said sample and, optionally, one or more clinical parameters of the subject are analyzed and used in conjunction with normalized level of PPA to determine the aggressiveness of prostate cancer in the subject.

In one aspect, a method for predicting the probability of upstaging or biochemical recurrence of prostate cancer in a subject, such as a patient diagnosed with prostate cancer, is provided. In one embodiment, said method comprises a) contacting a sample taken from said subject with a labeled prostate cancer specific peptide that is a substrate of PSA, b) determining the level of PPA in said sample; c) normalizing said level of PPA to (i) sPSA of said subject, (ii) said sample volume, (iii) combination of sPSA and said sample volume, and (iv) log transformed counterparts of any of i through iii; d) utilizing said normalized level of PPA to predict the likelihood of upstaging or biochemical recurrence of said prostate cancer in said subject. In an embodiment of such a method, one or more additional molecular biomarkers in said sample and, optionally, one or more clinical parameters of the subject are analyzed and used in conjunction with normalized level of PPA to predict the likelihood of upstaging and biochemical recurrence of prostate cancer in the subject.

### DESCRIPTION OF THE FIGURES

FIGS. 1A and 1B show an AMC standard curve and a graph of pmol AMC released for various samples (shown in logarithmic scale), respectively.

FIG. 2 provides a graph of pmol AMC released for various samples (shown in logarithmic scale).

FIGS. 3A and 3B show a graph of the average level of mRNA of the biomarker TMPRSS2:ERG (a fusion gene) in PCa patients shown in logarithmic scale and grouped according to the risk level of said PCa patients and a graph of the average level of mRNA of the molecular biomarker TXNRD2 in PCa patients, respectively.

FIGS. 4A and 4B show the ROC curve for a multi-variate analysis of the probability of Biochemical Recurrence after Surgery, a strong measure of PCa aggressiveness for baseline biomarkers and baseline supplemented with PPA and TXNRD2 molecular biomarkers, respectively.

### DETAILED DESCRIPTION OF THE INVENTION

The National Comprehensive Cancer Network (NCCN) recently established guidelines for assessing prostate cancer aggressiveness. These guidelines rely on clinical data, such as biopsy, stage, Gleason score, serum PSA results and the age of the patient and provide an assessment of aggressiveness and a recommendation for active surveillance. The latent form of prostate cancer often gets characterized as a low (favorable) risk prostate cancer and the aggressive form as a high (unfavorable) risk prostate cancer. Frequently, prostate cancer patients choose to undergo radical prostatectomy despite their cancer being characterized clinically as low risk. Following post-operative analysis of the pathology of the excised gland, Gleason scores are sometimes upgraded from the clinical value, and cancers are sometimes upstaged from the clinically established stage of the cancer predicted by biopsy, i.e., the results of the biopsy and clinical examination did not accurately reflect the severity of the cancer. This increased incidence of upgrading and upstaging suggests that there is a subset of patients that are clinically classified as non-aggressive, but pathology following surgery would characterize them as aggressive. This subset of patients is in need of more reliable aggressiveness test as well as a way to predict the risk of upstaging or upgrading.

Current NCCN guidelines (see “NCCN Guidelines for Patients®|Prostate Cancer”, National Comprehensive Cancer Network), use pre-surgery data for risk stratification that guides treatment options, however Gleason Score misclassifications due to biopsy sampling errors confound the guidelines. In addition, based on these NCCN guidelines, a classification of the different stages of clinical or pathology assessment is provided that differentiates clinical classification (T) from pathology classification (pT). The former (T) is based on pre-surgical clinical parameters such as biopsy, DRE and other imaging methods, whereas the latter (pT) is based on histopathological analysis post-surgery, most often following prostatectomy (full or partial). In most cases, researchers generally consider a patient to have been upstaged if there is an increase in clinical stage from T1-T2c to pathology stage pT3 after surgery.

It has been shown that the average value of PPA for a cohort that had no classification change post-surgery is higher than the average value of PPA for a cohort that was upstaged and upgraded (see for example The Prostate 73: 1731-1737). The latter cohort suggests that the overall cancer stage was indeed more aggressive than it was initially clinically assumed, and indicates that watchful waiting would not have been a viable option for those patients as it may have appeared to be from the clinical evaluation. Any one of the methods provided herein may be used for predicting the risk of upstaging or biochemical recurrence of prostate cancer. In addition, any one of the methods provided herein may be used for determining the aggressiveness of prostate cancer in a subject. Such methods may confirm the indolent nature of PCa in some subjects and can help reduce overtreatment. Any one of the methods provided herein may also be used to help overcome the need for restriction of screening, and its potential consequences of increased mortality, due to missed diagnosis of cancers. Importantly, the methods provided herein can be used as a supportive tool for patients and physicians to confidently recommended active surveillance instead of clinical intervention. Preliminary data suggests that 22% (14 out of 50) of patients diagnosed with non-aggressive PCa could have averted or delayed radical prostatectomy. Given the serious potential side-effects associated with a clinical intervention (e.g., incontinence, impotence, bowel dysfunction, osteoporosis, weakness, depression), the methods provided herein can be used as a tool that can aid the risk-benefit analysis of an informed treatment decision. Accordingly, the methods provided herein may not only assure patients that there is low risk for long-term harm without treatment but could also substantially reduce the healthcare costs associated with radical clinical interventions. These are estimated today to be about 14 billion dollars of healthcare costs associated with PCa annually, with an average of $5,000 per person.

Provided herein are methods for predicting the outcome of prostate cancer, such as in a subject diagnosed with prostate cancer, using certain parameters in a multi-variate analysis. Also provided are methods for determining sets of parameters that provide improved predictive power in assessing prostate cancer in a subject. As provided herein, “predictive power” refers to the ability to use the set of parameters in a multi-variate analysis to predict the outcome of prostate cancer in a subject. The predictive power can be determined with multi-variate analyses as described herein and/or known statistical methods. In some embodiments, the predictive power can be compared to the predictive power of other parameters or set of parameters. For example, results of one or more multi-variate analyses can be compared to “baseline” values, such as baseline cutoff values. “Baseline” refers to values that are indicative of the predictive power of another parameter or set of parameters to which results of a multi-variate analysis as provided herein may be compared. In some embodiments of any one of the methods provided herein, when such models include Receiver Operating Characteristic (ROC) curve analysis, the area under the curve (AUC) is a strong indicator of predictive power, with predictive power increasing as AUC values approach 1.

It has been surprisingly found that levels of PPA can be combined with levels of one or more other molecular biomarkers in multi-variate analyses to provide for the improved prediction of the outcome of prostate cancer in a subject. In some embodiments, the methods to predict the outcome may be used to make better informed clinical decisions, prior to treatment, to predict the potential for upstaging and/or biochemical recurrence of PCa after definitive treatment with curative intent, or to monitor prostate cancer in a subject. One aspect of the invention focuses on improved methods for optimal sample preparation, data collection, standard curve generation, and/or statistical analysis to make informed clinical decisions.

The disclosure provides methods that better predict the outcome of prostate cancer in a subject. In one embodiment of any one of the methods provided herein, predicting the outcome of prostate cancer includes determining the aggressiveness of, determining the prognosis of, determining the progression of, predicting the probability of upstaging or biochemical recurrence of prostate cancer, such as after prostate cancer therapy (e.g., including, but not limited to, chemotherapeutic treatment and radiation treatment, including brachytherapy and external beam radiation, as well as other types of radiation or beam therapies). In one embodiment of any one of the methods provided herein, the method may be used to determine the presence or absence of prostate cancer.

In some embodiments of any one of the methods provided herein, the methods include measuring the enzymatic activity of PSA in samples from subjects, such as patients, and measuring the genetic expression of one or more molecular biomarkers. The enzymatic activity of PSA may be normalized with one or more normalization factors to produce one or more normalized PPA values. “Normalized” as used herein refers to the standardization of data for analysis and is known to those of ordinary skill in the art. In some embodiments of any one of the methods provided herein the level is normalized by one of the normalization factors, such as by dividing a value for the level with a value for the normalization factor, such as a value for the level of the normalization factor. In some embodiments of any one of the methods provided herein, the value for the normalization factor can be one that is a measure of the normalization factor and that has been transformed with one or more numerical transformations (e.g., adding a constant value and/or multiplying by a factor or an inverse of the factor), and it is this transformed value for the normalization factor that is used to normalize the level, such as by dividing the level with the transformed value of the normalization factor. For example, normalization factors that may be used to normalize the level of enzymatic activity of PSA include, but are not limited to, expressed prostate secretion (EPS) sample volume, log of the EPS sample volume, serum PSA (sPSA) in the same or different sample from the subject, and/or log of the serum PSA (sPSA), PSA RNA, such as copy number, in the same or different sample from the subject, log of PSA RNA, such as copy number, RNA, such as amount, in the same or different sample from the subject, log of RNA, such as amount in the sample, and combinations thereof. In some embodiments of any one of the methods provided herein, the logarithm of the normalized PPA level is determined and used in the analysis. In general, a normalized PPA level for each normalization factor selected may be determined and/or used in the multi-variate analysis.

The level of genetic expression of one or more molecular biomarkers may also be normalized by a normalization factor to produce a normalized level of the genetic expression of the one or more molecular biomarkers in any one of the methods provided herein. The normalization factor may be any one of the normalization factors provided herein. For example, the level of genetic expression of one or more molecular biomarkers may be normalized by expressed prostate secretion (EPS) sample volume, log of the EPS sample volume, serum PSA (sPSA) in the same or different sample from the subject, log of the serum PSA (sPSA), RNA, such as amount, in the same or different sample from the subject, log of RNA, such as amount in the sample, etc., and combinations thereof. In some embodiments of any one of the methods provided herein, the logarithm of the normalized level of genetic expression of one or more molecular biomarkers is determined and used in the analysis. In general, a normalized level of genetic expression for each normalization factor selected may be determined.

In some embodiments of any one of the methods provided herein, at least one of the level of enzymatic activity of PSA and one or more normalized levels of enzymatic activity of PSA; and at least one of the level of genetic expression of one or more molecular biomarkers and one or more normalized levels of genetic expression of one or more molecular biomarkers may be used to predict the outcome of prostate cancer and/or prostate cancer therapy. For instance in any one of the methods provided herein, the level of enzymatic activity of PSA and the level of genetic expression may be used to predict the outcome of prostate cancer and/or prostate cancer therapy. In some embodiments of any one of the methods provided herein, at least two (e.g., at least three of, at least four of) of the level of enzymatic activity of PSA and one or more normalized levels of enzymatic activity of PSA and at least two of (e.g., at least three of, at least four of) the level of genetic expression of one or more molecular biomarkers and one or more normalized levels of genetic expression of one or more molecular biomarkers may be used to predict the outcome of prostate cancer and/or prostate cancer therapy. For instance in any one of the methods provided herein, the level of enzymatic activity of PSA, the level of enzymatic activity of PSA normalized by EPS, the level of genetic expression of one or more molecular biomarkers, and the level of genetic expression of one or more molecular biomarkers normalized by EPS may be used to predict the outcome of prostate cancer and/or prostate cancer therapy.

In some embodiments of any one of the methods provided herein, the at least one of the level of enzymatic activity of PSA and one or more normalized levels of enzymatic activity of PSA and at least one of the level of genetic expression of one or more molecular biomarkers and one or more normalized levels of genetic expression of one or more molecular biomarkers may be used as input values for a multi-variate analysis. For example in any one of the methods provided herein, a level of prostate specific antigen (PSA) proteolytic activity (PPA), one or more normalized levels of PPA, the level of genetic expression of the biomarker TMPRSS2:ERG (a fusion gene), and one or more normalized levels of genetic expression of the biomarker TMPRSS2:ERG may be used. As another example in any one of the methods provided herein, a level of prostate specific antigen (PSA) proteolytic activity (PPA), one or more normalized levels of PPA, the level of genetic expression of the biomarker TXNRD2, and one or more normalized level of genetic expression of the biomarker TXNRD2 may be used. In some instances of any one of the methods provided herein, the level of prostate specific antigen (PSA) proteolytic activity (PPA), one or more normalized levels of PPA, the level of genetic expression of the biomarker TMPRSS2:ERG, one or more normalized levels of genetic expression of the biomarker TMPRSS2:ERG, the level of genetic expression of the biomarker TXNRD2, and one or more normalized levels of genetic expression of the biomarker TXNRD2 may be used.

In general, in order to determine the predictive value of a set of parameters, multi-variate analyses using a number of statistical models can be employed to determine the best predictive model for the outcome of prostate cancer and/or prostate cancer therapy with the set of parameters. Values such as cutoff values may also be determined and used to predict the outcome of prostate cancer in a subject or to assess the predictive power of a particular multi-variate analysis. In some embodiments, to select a particular multi-variate analysis model for use in any one of the methods provided herein, input values may be entered into statistical software that determines the best fit from the data from a number of (e.g., greater than or equal to about 16,000) known models. The best fitting model(s) may be selected. In some instances, at least a portion of the known models may be selected. Once a suitable model is selected, a cutoff value for predicting the outcome of prostate cancer and/or prostate cancer therapy may be determined. The cutoff value may serve as a threshold for predicting the outcome of prostate cancer in the subject. For example, the cutoff value may indicate whether a subject is expected to have a poor outcome, the subject is in a low risk or high risk category, the cancer is aggressive or not aggressive, the subject has poor prognosis, the subject requires further monitoring, the subject is likely to be upgraded or upstaed, the subject is likely to experience biochemical recurrence, etc. As another example the cutoff value may also be used to assess the predictive power of a particular multi-variate model and/or set of parameters.

In some embodiments of any one of the methods provided herein, the multi-variate analysis comprises providing input values (e.g., information derived from a patient and/or sample (e.g., level of enzymatic activity of PSA, normalized level of enzymatic activity of PSA, level of genetic expression of a molecular biomarker, normalized level of genetic expression of a biomarker)) into a model (e.g., algorithm or equation) and generating results, including one or more values from the model. In some embodiments of any one of the methods provided herein, the values include cutoff value(s). In some instances, the information provided may be the same as the information used to select the multi-variate analysis model for use in performing any one of the methods or predicting outcome as provided herein. The results, including the values, produced by the model may be compared to a cutoff value to predict the outcome of prostate cancer and/or prostate cancer therapy in any one of the methods provided herein. Less that the cutoff value can indicate poor prognosis, while greater than the cutoff value indicates good prognosis.

In general, any suitable input values (e.g., information derived from a patient and/or sample) may be used. Non-limiting examples of input values include any one or more of the parameters of Tables 1-4 provided herein. In some embodiments, of any one of the methods provided herein the input value(s) are those of any one of Tables 1-4.

Sample Collection and Preparation

The present methods involve testing samples for PPA. By “sample” herein is meant a sample containing isoforms of PSA of various enzymatic activity that is correlated to prostatic disease, including, but not limited to, urine, semen, prostatic fluid, seminal vesicle fluid, prostate tissue samples (for example biopsy sample(s) (e.g., homogenized tissue samples), expressed prostatic secretions (EPS), and post prostatic massage urine.

PSA reaches the serum after diffusion from luminal cells through the epithelial basement membrane and prostatic stroma, where it can pass through the capillary basement membrane and epithelial cells or into the lymphatics. (Sokoll et al. 1997). PSA can also be isolated from body fluids including, but not limited to, semen, seminal plasma, prostatatic fluid, serum, urine, urine after prostate massage, and ascites.

In some embodiments of any one of the methods provided, the sample is urine. In some cases, standard urine is collected, either “first catch” urine or total samples. In some embodiments of any one of the methods provided, urine samples are collected after standard DRE prostatic massage, which are referred to herein as “post prostatic massage urine” (PMU).

In other embodiments of any one of the methods provided, the test sample is semen, seminal fluid or seminal plasma. Seminal plasma can be obtained by allowing semen to liquefy for one hour at room temperature followed by centrifugation 1000 g at 4° C. for ten minutes. See e.g., Edstrom A. et al. J. Immunol. 181, 3413-3421 (2008).

In other embodiments of any one of the methods provided, the test sample is prostatic fluid. Prostatic fluid can be obtained following a prostatic massage, e.g., post Digital Rectum Exam (DRE) and either milking the urethra directly (called Expressed Prostatic Secretions, EPS), or in post-DRE urine, or following radical prostatectomy by squeezing the excised prostate gland. In some embodiments, using prostatic fluid collected clinically as EPS is preferred, as post-DRE urine introduces complications to the fluorescent measurement, as it auto-fluoresces. These complications can be avoided if prostate fluid is used as the test sample since (our studies show) it does not auto-fluoresce.

In serum, total PSA (tPSA) levels represent the combined concentrations of several free isoforms (fPSA) and protease-inhibitor complexes (cPSA) that can be recognized by immunoassay.

In some embodiments, blood, serum and/or plasma may be used, although in some embodiments, these samples are not preferred.

The samples can be tested either “straight” or directly, with no sample preparation, or with some sample preparation. As will be appreciated by those in the art, a number of sample preparation methods may be utilized, including the removal of cells or non-protease proteins, and buffers (e.g., the addition of high salts, etc.), reagents, or assay components, etc., added.

Many types of samples can be used with the methods provided herein. There are several methods for collecting prostatic fluid (PF) from patients at various stages of the diagnostic cycle to provide samples of particular use, including:

i. PF collected as post digital rectal exam (DRE) urine—Referred to as post prostatic massage urine (PMU), which contains an amount of PF. Such samples can be centrifuged to yield both a PMU pellet and supernatant.

ii. PF collected by milking the prostate gland following radical prostatectomy (PF)

iii. PF collected by milking the gland prior to surgery under anesthesia—Referred to as expressed prostatic secretions (EPS) under pre-surgery anesthesia (EPS_{A})

iv. PF collected by milking the gland of patients undergoing biopsy (EPS_{B})

v. PF collected by milking the gland using an attentive DRE during a routine visit, under conscious sedation or not (EPS_{V}) and

vi. PF collected in semen (S-EPS).

EPS and PMU samples are typically collected by urologists with a DRE under conscious sedation during a biopsy visit. It is important to note that EPS samples are currently widely collected from males during the diagnosis of urinary infection, with the collection technique being widely used by practicing urologists.

As will be appreciated by those in the art, samples may be treated or prepared upon collection in a variety of ways. One example of a sample preparation method is to centrifuge the sample at relatively low speed, measure the volume of the sample (e.g. by drawing it up into a micropipette), further dilute the sample with PBS, centrifuge the sample/buffer again, aliquot the supernatant, and store the aliquots of supernatant and remaining pellet until analysis can be performed. In some embodiments, storage condition for samples is −80° C. In some embodiments, storage conditions for samples include limited freeze-thaw cycles.

Screening Samples for PPA

The PPA assay measures the activity of PSA rather than the amount of protein. PSA is a 30 kDa serine protease enzyme secreted by prostate epithelial cells. It is first produced as an inactive zymogen. Subsequently, signal peptidases cleave the 17 amino acid pre-sequence intracellularly and an additional 7 amino acid pro-sequence is removed in the extracellular environment. This resulting activation allows PSA to catalyze the cleavage of substrates that have a glutamine at the P1 position. The PPA assay may utilize this function to measure the PPA present in various samples.

The assessment of the enzymatic function of PSA in physiological fluids can use a synthetic substrate in a regulated in vitro environment, by providing high excess of a labeled synthetic peptide substrate in vitro, in some embodiments. This synthetic peptide can be a substitute model for physiological substrates, such as naturally occurring proteins including macroglobulins and semenogelin. The peptide substrate can provide a means of measuring PSA's efficacy as a peptidase in a given physiological sample under standardized conditions, after controlling the ionic activity, the pH and temperature. Examples of the substrates used for the PSA Peptidase Activity include, but are not limited to, HSSKLQ-Tag (SEQ ID NO: 16) or HSSK-Hic-Q-Tag (SEQ ID NO: 17), where Tag represents a label. In some embodiments of any one of the methods provided, this label is the AMC (7-amino methyl coumarin) and the products are HSSKLQ (SEQ ID NO:1) and HSSK-Hic-Q (SEQ ID NO: 14) respectively. In this context, HIV stands for hydroxyl-isovaleric and HIC stands for hydroxyl-isocaproic. The measurement of the proteolytic function of the enzyme can be assessed by measuring the fluorescent signal generated by the cleaved fluorogenic tag.

By “peptides” or grammatical equivalents herein is meant proteins, polypeptides, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L configuration.

When the peptide is used as a substrate during the assay, e.g., as a PCSP, the peptide can contain both naturally occurring and peptidomimetic structures, as long as the peptidomimetic residues of the PCSP do not interfere with the cleavage of the peptide and/or the correlation of activity to the diagnosis or prognosis.

As discussed below, when the protein is used as a capture substrate it may be desirable in some embodiments to utilize protein analogs to retard degradation by sample contaminants, although in many embodiments capture peptides utilizing native amino acids are used.

Provided herein are exemplary substrate peptides that are PCSPs and that can be used in any one of the methods provided herein. By “prostate cancer specific peptide” or “PCSP” or “prostatic disease specific peptide” or grammatical equivalents herein is meant a peptide whose cleavage by one or more proteases in a sample is correlated to prostate cancer and disease. In some embodiments, as is more fully outlined below, the PCSP is specific to PSA in the context of the assay. That is, the specificity of the peptide for the protease may be altered depending on what other proteases are present; for example, in general, semen contains more proteases that urine, and thus the absolute specificity of the peptide may be less for urine.

The substrates that may be used in the methods provided depend on the target enzyme. In some embodiments of any one of the methods provided, the enzyme is PSA, as is more fully described below. In the case of PSA, a peptide that finds particular use is the peptide HSSKLQ (SEQ ID NO:1), wherein cleavage occurs after the glutamine (Q); see Denmeade et al., Cancer Research 57:4924 (1997), incorporated by reference in its entirety. As outlined below, the PCSPs of any one of the methods provided can be conjugated to labels, including optical (fluorescent) and electrochemical labels, to allow for detection of cleavage. In addition to the HSSKLQ (SEQ ID NO:1) peptide, a number of other peptides are PCSPs and may be used in any one of the methods provided. These peptides include, but are not limited to, KGISSQY (SEQ ID NO.2), SRKSQQY (SEQ ID NO. 3), GQKGQHY (SEQ ID NO. 4), EHSSKLQ (SEQ ID NO. 5), QNKISYQ, (SEQ ID NO. 6), ENKISYQ (SEQ ID NO. 7), ATKSKQH (SEQ ID NO. 8), KGLSSQC, (SEQ ID NO. 9), LGGSQQL (SEQ ID NO. 10), QNKGHYQ (SEQ ID NO. 11), TEERQLH (SEQ ID NO. 12), GSFSIQH (SEQ ID NO. 13), SKLQ (SEQ ID NO. 15), as well as analogs.

Such peptides, as well as other enzyme-cleavable peptides, including peptides containing substitute, modified, unnatural or natural amino acids in their sequences, as well as peptides modified at their amino or carboxy terminus, can be made from their component amino acids by a variety of methods well known to ordinarily skilled artisans, and practiced thereby using readily available materials and equipment, (see, e.g., The Practice of Peptide Synthesis (2^{nd} Ed.), M. Bodanskzy and A. Bodanskzy, Springer-Verlag, New York, N.Y. (1994), the contents of which are incorporated herein by reference).

In some embodiments of any one of the methods provided, fluorogenic PCSPs are utilized. There are a number of fluorogenic groups that can be used in the determination of protease cleavage, including, but not limited to, AMC (7-Amino-4-methylcoumarin); MCA ((7-Methoxycoumarin-4-yl)acetyl), p-nitroanilide (pNA), etc.

In addition to fluorogenic substrates relying on a single fluorophore which is activated by cleavage, fluorescence resonance energy transfer (FRET also known as non-radiative energy transfer or Forster energy transfer) systems can also be used. In these embodiments, a fluorophore reporter and a quencher is used, with the protease cleavage site between the two. As one specific example, the quenching moiety may be a dye molecule capable of quenching the fluorescence of the signal fluorophores via the well-known phenomenon of FRET. In FRET, an excited fluorophore (donor dye; in this instance the signal fluorophore) transfers its excitation energy to another chromophore (acceptor dye; in this instance the quencher). Such a FRET acceptor or quencher may itself be a fluorophore, emitting the transferred energy as fluorescence (fluorogenic FRET quencher or acceptor), or it may be non-fluorescent, emitting the transferred energy by other decay mechanisms (dark FRET quencher or acceptor).

Examples of signal and quencher labels that are FRET dye pairs are well known in the art, see for example, Marras et al., 2002, Nucleic Acids Res., 30(21) e122; Wittwer et al., 1997, Biotechniques 22:130-138; Lay and Wittwer, 1997, Clin. Chem. 43:2262-2267; Bernard et al., 1998, Anal. Biochem. 255:101-107; U.S. Pat. Nos. 6,427,156; 6,140,054 and 6,592,847, the disclosures of which are incorporated herein by reference.

In addition, fluorescent based detection systems as discussed above can be done as “solution phase” assays as will be readily appreciated by those in the art. Alternatively, the PSA enzymatic activity tests using fluorescence can be done as “solid support” assays as well. Thus, for example, either a peptide labeled with a single fluorophore as described above or a dual labeled FRET peptide can be attached to a solid support and a test sample can be added and fluorescence monitored.

Other peptides can be used as the capture substrates (e.g., the “PSA peptide”) for use in the assay systems described herein. For example, PSA cleaves with some specificity the peptide HSSKLQ (SEQ ID NO:1) relative, for example, to chymotrypsin. Depending on the test sample, less specific peptides can be used. As will be appreciated those in the art, there are a number of optical (e.g., including fluorescence based) assays that can be run on peptide-based substrates. In general, these rely on optical changes, for example fluorescence, that occur upon cleavage, as generally described above.

Other PSA substrates include naturally occurring substrates such as semenogelin I, semenogelin II, fibronectin, laminin, insulin-like growth factor binding proteins, the single chain form of urokinase-type plasminogen activator and parathyroid hormone related protein. In general, the cleavage of these PCSPs are correlated to the presence of particular proteases in the samples. Proteases represent a number of families of proteolytic enzymes that catalytically hydrolyze peptide bonds. By “protease” or “proteinase” herein is meant an enzyme that can hydrolyze proteins by hydrolysis of the peptide (amide) bonds that link amino acids. Principal groups of proteases include serine proteases, cysteine proteases, aspartic proteases and metalloproteases.

Certain inventive embodiments provide, at least in part, for the assay of proteases, particularly prostate specific antigen (PSA) serine protease, in the samples. That is, in some embodiments of any one of the methods provided, the activity of PSA in the sample such as post prostatic massage urine or expressed prostatic secretions is assayed using any one of the substrates provided herein that is both cleaved by PSA and is not cleaved by other proteases in the particular sample.

In some conventional systems, this enzymatic assay can be performed in a 96 well plate and can use a standard laboratory microplate reader. Because active PSA (aPSA) cleaves glutamine residues, a PSA specific peptide sequence ending in glutamine (Mor-HSSKLQ-AMC; SEQ ID NO: 18) is labeled with AMC (7-amino-4-methyl coumarin), a fluorescent dye. AMC does not fluoresce when attached to the peptide, but does fluoresce when it has been separated from the peptide by enzymatic activity of PSA. This tagged peptide sequence can be added to wells containing various known concentrations of aPSA to create a PSA standard curve. As the aPSA enzyme recognizes the peptide substrate, the fluorescent dye is released, and the amount released can be measured over time using an excitation wavelength of 380 nm and an emission wavelength of 450 nm. The fluorescence values and time can be used to create a series of slopes, which ultimately provide an equation to calculate the PPA present in a sample. In certain embodiments, each dilution for the standard curve and sample is run in triplicate.

PPA values may be evaluated against a control or threshold value, wherein said control or threshold value is determined by obtaining PPA values, creating a Receiver Operating Characteristic (ROC) curve based on said PPA values, identifying the point on the ROC curve closest to the coordinates (0,1), using said point on said ROC curve to establish cutoff values, utilizing said cutoff values to help predict outcome.

Further improvements for the PPA assay over previous methods have been made and are described herein. To control for variations in different lots or supplies of the commercial aPSA used in the assay, a second standard curve using known concentrations of the fluorescent dye can also be used in any one of the methods provided herein. In addition, sample preparation is modified to minimize the inhibitory effect of PBS used as the storage buffer for samples. Specifically, in some embodiments of any one of the methods provided herein, the samples are removed from the −80° C. freezer and left to thaw for 15 minutes at room temperature. After mixing the sample with equal volume of Buffer AA (0.1M Tris-HCl, pH 7.5, 3M NaCl, and 0.4% BSA), 50 μL can be loaded in triplicate onto a microplate (e.g., 96-well microplate) followed by the addition of 50 μL of peptide solution, 0.8 mM peptide in Buffer A (0.05 mM Tris-HCl, pH 7.5, 1.5M NaCl, and 0.2% BSA). A serial dilution of aPSA (25-3156 ng/mL) in PBSAA (mix of equal volume of PBS and Buffer AA) and a serial dilution of AMC (50-3214 pmol/well) can be run concurrently with the samples to generate standard curves for both aPSA and AMC. The microplate loaded with standards and samples can be placed in the microplate reader within 30 minutes of sample removal from the freezer and run in top read fluorescence mode (380 nm excitation/450 nm emission) at 30° C. for 40 minutes with a reading collected every 2 minutes. The fluorescence intensity data collected in triplicate can be averaged, plotted as relative fluorescent units (RFU) over time, from which slope can be calculated through linear regression analysis. A standard curve can be generated by plotting known active PSA (aPSA) concentration as a function of the slope generated and fitting with a bi-normal curve. The AMC standard curve can be generated by plotting RFU as a function of the amount of AMC (pmole) and fitting with a linear regression. Using the equation from the two standard curves, PPA in a sample can be calculated and expressed as either aPSA in μg/mL or pmol AMC released/minute/mL.

Screening Samples for Other Biomarkers

Level(s) of gene expression of one or more molecular biomarkers can also be obtained or determined in any one of the methods provided herein, including DNA or RNA levels of any one or more of TXNRD1, TXNRD2, GAPDH, PSA, TMPRSS2:ERG and PCA3. As used herein, the “level of gene expression” is any value indicative of the amount of an expression product, DNA or RNA, of the gene of interest. Methods for determining levels of such resultant products in a sample are well known to those of ordinary skill in the art.

Assays for detecting mRNA include, but are not limited to, Northern blot analysis, RT-PCR, sequencing technology, RNA in situ hybridization (using e.g., DNA or RNA probes to hybridize to RNA molecules present in the sample), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface (e.g., a glass wafer) with addressable locations, such as an Affymetrix microarray (Affymetrix®, Santa Clara, Calif.)). Methods for designing nucleic acid binding partners, such as probes, are well known in the art.

In some embodiments of any one of the methods provided herein, quantitative real time polymerase chain reaction (qRT-PCR) can be used for molecular biomarker screening. For example, RNA can be prepared with the RNEasy MiniKit by Qiagen qRT-PCR methods as described in Clark et. al. Clinical Chemistry 2008, 54 (12), 2007-2017. The levels of the one or more molecule biomarkers may also be normalized as provided herein and used in the multi-variate analysis of any one of the methods provided herein.

Assays for detecting protein levels include, but are not limited to, immunoassays (also referred to herein as immune-based or immuno-based assays, e.g., Western blot and ELISA), Mass spectrometry, and multiplex bead-based assays. Binding partners for protein detection can be designed using methods known in the art. Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

Normalization

Other measurements may be carried out for inclusion as an additional parameter or for normalization in any one of the methods as provided herein, including the determination of EPS volume, actual amounts of total PSA (tPSA) and free PSA (fPSA) in a sample and in serum (sPSA). In some embodiments, these additional measurements may be done using commercial ELISA kits. The measured values may be used for normalization of PPA and/or the level of gene expression of one or more molecular biomarkers as provided herein. For normalization of molecular biomarkers, the total RNA in a sample may also be measured.

In addition, the normalized measures may be further converted into logarithm values in any one of the methods provided herein (e.g., Table 1). Each measure of PPA and/or molecular biomarkers without or with normalization in various ways is considered a variable for analysis in any one of the methods provided herein.

Statistical Analysis

Initial data assessment may be carried out using graphical trends and distributional properties for PPA or other parameters with respect to existing criteria. A receiver operating characteristic (ROC) analysis may be performed to assess which of the several variables have the highest discriminatory power for predicting the outcome of prostate cancer, such as the presence of aggressive PCa. Such an analysis may be done using statistical software such as JMP (e.g. version 10.0), R, SAS® (e.g. version 9.2). In addition to the level of PPA alone, its normalized counterparts (e.g., ratio of PPA over sPSA, or EPS volume), or logarithmic versions of either the levels or normalized counterparts may also considered and used in any one of the methods provided herein. Other parameters that may be used in any one of the methods provided herein include age, Gleason score, serum total PSA (sPSA), EPS volume, etc. For each parameter, an empirical ROC curve may be estimated along with a corresponding area under the curve (AUC). The ROC curves may be compared using a chi-square test based on differences in the areas under the empirical ROC curves. Estimated odds ratios from two separate logistic regression analyses suitable for an unmatched case-control study may be computed in order to determine the extent to which PCa pathology is associated with the parameter, such as PPA independently or adjusted for any of normalization factors, or set of parameters as provided herein. Test may be two-tailed and considered as statistically significant at P<0.05 for the null hypothesis. Predictive models may be selected using a generalized linear model (GLIM) as well as a logistic regression analysis to predict the outcome of prostate cancer, such as the probability of aggressive Pca, as a function of the level of PPA, or its normalized or logarithmic values, alone or in combination with other parameters as provided herein, such as the level of gene expression of one or more molecular biomarkers (e.g., RNA levels of any one or more of the genes provided herein).

Endpoints may be determined as follows. First a dichotomous variable of interest may be chosen. Upstaging to pT3 after surgery is dichotomous in that it occurs (Yes) or it does not (No). Similarly, biochemical recurrence defined as a rise in the serum PSA value above 0.2 ng/mL within a 2.5 years follow-up period either occurs (Yes) or it does not (No).

### EXAMPLES

Example 1: shows a series of variables and their normalized counterparts that were assembled for nominal logistic regression analysis modeling. A representative set of PPA variables (including other related criteria) used to model upstaging to pT3 is given in Table 1.

TABLE 1
Definition of variables

| Variables | Description |
|---|---|
| Expressed Volume (uL) | Initial volume of prostatic |
| | excretion post massage |
| PreBx PSA | The level of serum PSA found |
| | in patient prior to biopsy |
| | of prostate tissue, collected |
| | prior to surgery |
| PPA PSA ACT | Peptidase activity function |
| | measured as a turnover rate |
| | of a substrate in a |
| | diluted sample |
| PSA ACT sPSA | Normalized PSA peptidase |
| | activity (PPA) to the |
| | serum PSA value, |
| | constituting the ratio PSA |
| | described in the paper |
| Age | Age of patient at |
| | time of surgery |
| PreTx Gleason Score | Gleason score based on |
| | biopsy tissue collected |
| | clinically |
| Normalized_PSA_ACT_EPS_Vol | PPA PSA ACT/[Expressed |
| | Volume/(3000 + |
| | Expressed Volume)] |
| Norm_PSA_ACT_sPSA_EPS_Vol | PSA ACT sPSA/[Expressed |
| | Volume/(3000 + |
| | Expressed Volume)] |
| InvNormalized_PSA_ACT_EPS_Vol | PPA PSA ACT/[Expressed |
| | Volume*(3000 + |
| | Expressed Volume)] |
| InvNorm_PSA_ACT_sPSA_EPS_Vol | PSA ACT sPSA/[Expressed |
| | Volume*(3000 + |
| | Expressed Volume)] |
| LNNormalized_PSA_ACT_EPS_Vol | Log(PSA ACT sPSA/ |
| | [Expressed Volume/ |
| | (3000 + Expressed |
| | Volume)]) or |
| | a constant if missing/0 |
| LNNorm_PSA_ACT_sPSA_EPS_Vol | Log(PPA PSA ACT/ |
| | [ExpressedVolume/ |
| | (3000 + Expressed |
| | Volume)]) or a |
| | constant if missing/0 |
| LNInvNormalized_PSA_ACT_EPS_Vol | Log(PPA PSA ACT/ |
| | [Expressed |
| | Volume*(3000 + |
| | Expressed Volume)]) |
| LNInvNorm_PSA_ACT_sPSA_EPS_Vol | Log(PSA ACT sPSA/ |
| | [Expressed |
| | Volume*(3000 + |
| | Expressed Volume)]) |

All combinations of logistic models were constructed for the upstaging, upgrading, up-risking or biochemical recurrence model using AIC (Akaike Information Criterion) for model selection (>16K models). The best two models for each endpoint were chosen for further validation. The Youden statistic J, representing the maximum distance from the chance diagonal in the ROC (receiver operating characteristics) curve, was chosen to determine the optimal cut point for the logistic model used in classification. The sensitivity, specificity, positive predictive value (PPV), negative predictive value (NPV), and area under the curve (AUC) of the ROC curve were compared against a model containing PreBx PSA and PreBx Gleason score in each cohort. The model containing PreBx PSA and PreBx Gleason score is referred to as the “Baseline” model.

Similar calculations were performed for the models of upstaging or recurrence which take additional variables into account. In those cases the variables considered include molecular biomarkers GAPDH, PSA, TXNRD1, TXNRD2, TMPRSS2:ERG, PCA3 and their normalized counterparts. A representative set of molecular biomarker variables used to model upstaging to pT3 is given in Table 2.

TABLE 2
Definition of molecular biomarker variables

| Variables | Description |
|---|---|
| NZLNPCA3_Ave | Log(PCA3 average) or a |
| | constant if missing/0 |
| NZLNPCA3_AVE_RNA | Log(PCA3 average, normalized |
| | by total ng into cDNA |
| | reaction) or a constant if missing/0 |
| NormalizedmPSA_Ave | Log(mPSA average) or a |
| | constant if missing/0 |
| NormalizedmPSA_Ave_RNA | Log(mPSA average, normalized by |
| | total ng into cDNA |
| | reaction) or a constant if missing/0 |
| NZLNmTRD1_Ave | Log(mTXNRD1) or a constant if |
| | missing/0 |
| NZLNmTRD1_Ave_RNA | Log(mTXNRD1, normalized |
| | by total ng into cDNA |
| | reaction) or a constant if missing/0 |
| NZLNmT2_Ave | Log(TMPRss2:ERG average) |
| | or a constant if missing/0 |
| NZLNmT2_Ave_RNA | Log(TMPRss2:ERG average, |
| | normalized by total ng into |
| | cDNA reaction) or a constant |
| | if missing/0 |

Note that the logistic models obtained can be used to construct nomograms linking probability in the model to the values of the tested parameters, or software can be written to produce a probability for a recently tested patient that would generate a value above or below the cutoff in a probability table generated by the model. The patient could then be advised on the need for definitive treatment based on the outcome of the test.

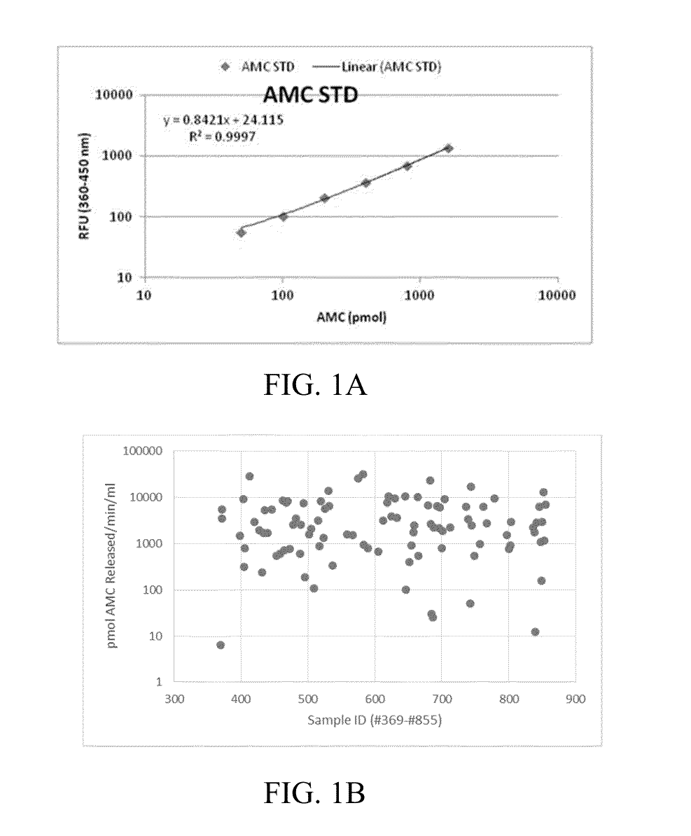

EXAMPLE 2 provides an example of measuring PPA in EPS samples from PCa patients using an AMC standard curve. A 2-fold serial dilution of AMC from 0.5 pmol/μL to 32 pmol/μL was made with Buffer A (0.05 mM Tris-HCl, pH 7.5, 1.5M NaCl, and 0.2% BSA). To each well of a 96-well microplate, 100 μL of each dilution was loaded (50-3214 pmol/well) along with 100 μL of Buffer A alone as a negative control. In parallel, samples were removed from the −80° C. freezer and left to thaw for 15 minutes at room temperature. After mixing the sample with equal volume of Buffer AA (0.1M Tris-HCl, pH 7.5, 3M NaCl, and 0.4% BSA), 5 μL was loaded in triplicate onto the microplate followed by the addition of 50 μL of peptide solution, 0.8 mM peptide in Buffer A (0.05 mM Tris-HCl, pH 7.5, 1.5M NaCl, and 0.2% BSA). The microplate was placed in the microplate reader and run in top read fluorescence mode (380 nm excitation/450 nm emission) at 30° C. for 40 minutes with a reading collected every 2 minutes. The fluorescence intensity data collected in triplicate was averaged, plotted as relative fluorescent units (RFU) over time, from which slope was calculated through linear regression analysis. The AMC standard curve was generated by plotting RFU as a function of the amount of AMC (pmole) and fitting with a linear regression. Using the equation from the AMC curve (FIG. 1A), PPA in a sample was calculated in pmol AMC released/minute/mL (FIG. 1B).

FIGS. 1A-1B depict a AMC standard curve, and it was used to estimate PPA in EPS samples from PCa patients. FIG. 1A shows an AMC standard curve. FIG. 1B shows PPA in 110 EPS samples measured in pmole AMC released/min/mL (shown in logarithmic scale).

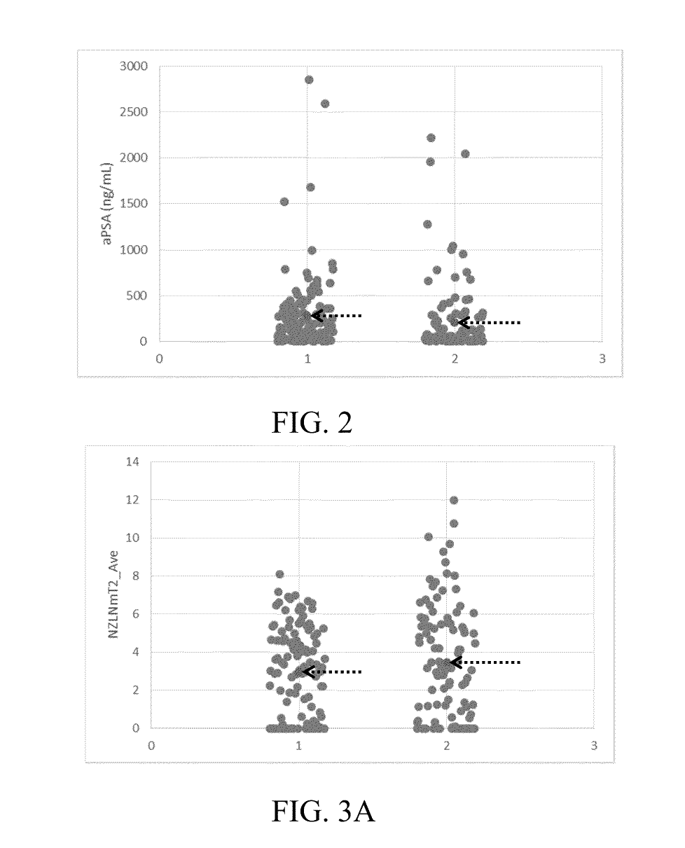

EXAMPLE 3 provides an example of measuring PPA in EPS samples from PCa patients using aPSA standard curve. A 2-fold serial dilution of purified PSA was prepared with PBSAA Buffer (equal volume mix of PBS and Buffer AA) covering the concentration range of 25-3156 ng/mL. 50 mL/well of each dilution was loaded in triplicate onto a 96-well microplate. The samples were removed from the −80° C. freezer and left on ice to thaw. After mixing the sample with equal volume of Buffer AA (0.1M Tris-HCl, pH 7.5, 3M NaCl, and 0.4% BSA), 50 μL of each sample was loaded in triplicate onto the same microplate followed by addition of 50 μL of the peptide solution (0.8 mM AMC labeled peptide (Mor-HSSKLQ-AMC; SEQ ID NO: 18) in Buffer A (0.05 mM Tris-HCl, pH 7.5, 1.5M NaCl, and 0.2% BSA)). The microplate was read by a standard plate reader at excitation wavelength of 380 nm and an emission wavelength of 450 nm to measure the signals from free AMC cleaved off by aPSA over 40 minutes at a 2-min interval. A standard curve of aPSA was generated by plotting aPSA concentration (ng/mL) as a function of slope (RFU/min). Using the equation, aPSA in an EPS sample was estimated. The PPA was normalized with serum PSA known for each patient (ng/mL) and plotted as a function of risk level as shown in FIG. 2. The average PPA/sPSA in low risk PCa patients is apparently higher than that in high risk patients.

FIG. 2 depicts the correlation of PPA in EPS samples with the risk level of PCa patients. PPA present in individual PCa patients was normalized with serum PSA and grouped according to the risk level of said PCa patients. Low risk was assigned with “1” and characterized as having pT2a, Gleason score≦6, and no evidence of clinical or biochemical tumor recurrence (2-5 year follow up). High risk was assigned with “2” and characterized as having PCa specific death on follow up, lymph node or distant metastases (N1, M1), seminal vesicle invasion (pT3b), and extracapsular tumor extension (pT3a). The average PPA in each risk group is shown with an arrow.

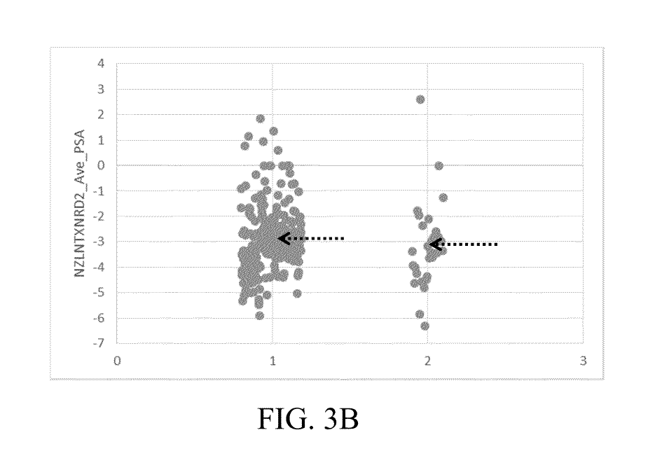

EXAMPLE 4 provides examples of measuring molecular biomarkers in EPS samples from PCa patients with qRT-PCR. The method for the detection of TMPRSS2:ERG fusion RNA is described in Clark, J. P. et al., Performance of a Single Assay for Both Type III and Type VI TMPRSS2:ERG Fusions in Noninvasive Prediction of Prostate Biopsy Outcome. Clinical Chemistry 2008, 54 (12), 2007-2017. Briefly, 200 ng of recovered RNA was converted to cDNA by reverse transcription. Once cDNA was prepared, a 25 uL reaction was prepared for quantitative PCR containing forward and reverse primers specific for the desired target (e.g., TMPRSS2:ERG or TXNRD2) along with a probe containing FAM at its 5′ end and black hole Quencher® dye at the other. FAM liberated by the exonuclease activity of HotStar Taq® DNA polymerase was quantified at each thermal cycle as fluorescence at 518 nm after excitation at 494 nm with the on-board fluorimeter in the RotorGene® thermal cycler. Quantification was obtained from a standard curve analyzed by the Ct (Cycle at Threshold) method. The expression level of the two genes in the PCa patients is shown in FIG. 3.

EXAMPLE 5 provides representative results of statistical analyses (Tables 3 and 4). Table 3 shows the results with PPA and its normalized counterparts. Table 4 shows the results with molecular biomarkers and other criteria. Upstaging was predicted for patients with a model probability of above or below the cutoff value. The Negative Predictive Value (NPV) of the test for patients with test scores below the cutoff value was a good indicator that a patient with a negative test score (i.e., one below the cutoff value) would be very unlikely to have an aggressive form of the disease that would preclude active surveillance. The Baseline model includes PreBx PSA and PreBx Gleason scores, where PreBx indicates pre-biopsy. Improvements over the baseline model are seen for some models containing PPA and some models containing molecular biomarkers. Not all molecular biomarkers showed improvement over baseline for the analyses performed.

TABLE 3
T Stage Upstage to pT3 or worse, 2012 cohort

| Model | N | Cutoff | AUC | Sens | Spec | PPV | NPV |
|---|---|---|---|---|---|---|---|
| Baseline | 218 | 0.050 | 0.768 (0.657, 0.878) | 0.79 | 0.64 | 0.13 | 0.98 |
| PPA.PSA.ACT PSA.ACT.sPSA Age | 218 | 0.096 | 0.813 (0.685, 0.941) | 0.71 | 0.85 | 0.24 | 0.98 |
| PreTx.GleasonScore | | | | | | | |
| LNInvNorm_PSA_ACT_sPSA_EPS_{—} | | | | | | | |
| Vol | | | | | | | |
| Age PreTx.GleasonScore | 219 | 0.072 | 0.766 (0.608, 0.923) | 0.71 | 0.78 | 0.18 | 0.98 |
| LNInvNormalized_PSA_ACT_EPS_{—} | | | | | | | |
| Vol | | | | | | | |

FIG. 3 shows the correlation of molecular biomarkers with the risk level of PCa patients. FIG. 3A provides data for the biomarker TMPRSS2:ERG (a fusion gene) in PCa patients. The average level of mRNA of said fusion gene is shown in logarithmic scale and grouped according to the risk level of said PCa patients. Low risk is assigned with “1” and characterized as having pT2a, Gleason score≦6, and no evidence of clinical or biochemical tumor recurrence (2-5 year follow up). High risk is assigned with “2” and characterized as having PCa specific death on follow up, lymph node or distant metastases (N1, M1), seminal vesicle invasion (pT3b), and extracapsular tumor extension (pT3a). The average value in each risk group is shown with an arrow. FIG. 3B provides data for the molecular biomarker TXNRD2 in PCa patients. The expression level of said molecular biomarker was normalized with the average PSA in the same sample, shown in logarithmic scale, and grouped according to biochemical recurrence. No biochemical recurrence is assigned as “1” whereas with biochemical recurrence is assigned as “2”. The average value in each group is shown with an arrow.

TABLE 4
T Stage Upstage to pT3 or worse, 2012 cohort

| Model | N | Cutoff | AUC | Sens | Spec | PPV | NPV |
|---|---|---|---|---|---|---|---|
| Baseline | 198 | 0.053 | 0.764 (0.652, 0.875) | 0.79 | 0.63 | 0.13 | 0.98 |
| Expressed Volume . . . uL. | 198 | 0.14 | 0.727 (0.570, 0.884) | 0.50 | 0.96 | 0.50 | 0.96 |
| Age | | | | | | | |
| PreTx Gleason Score | | | | | | | |
| NZLNmTRD1_Ave_RNA | | | | | | | |
| Expressed Volume . . . uL. | 198 | 0.08 | 0.800 (0.659, 0.942) | 0.71 | 0.84 | 0.25 | 0.97 |
| Age | | | | | | | |
| PreTx Gleason Score | | | | | | | |
| NZLNmTRD1_Ave_RNA | | | | | | | |
| NZLNmT2_Ave_RNA | | | | | | | |

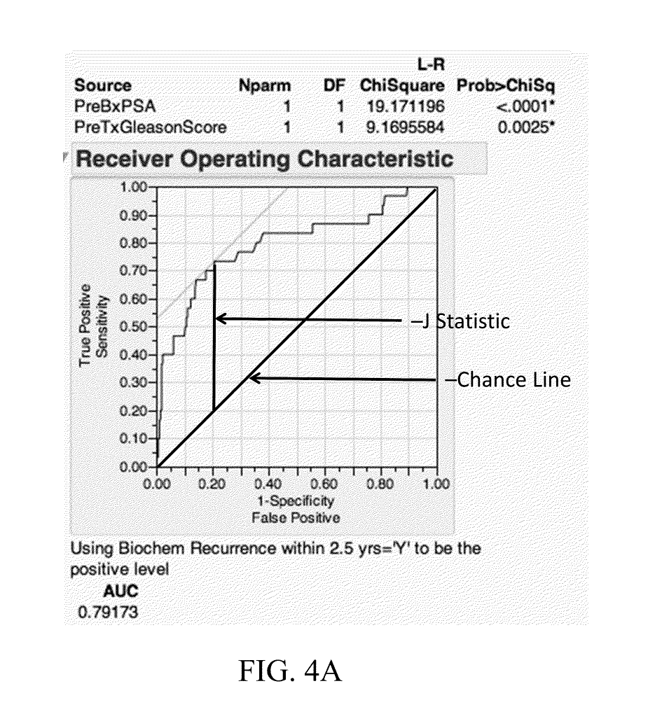

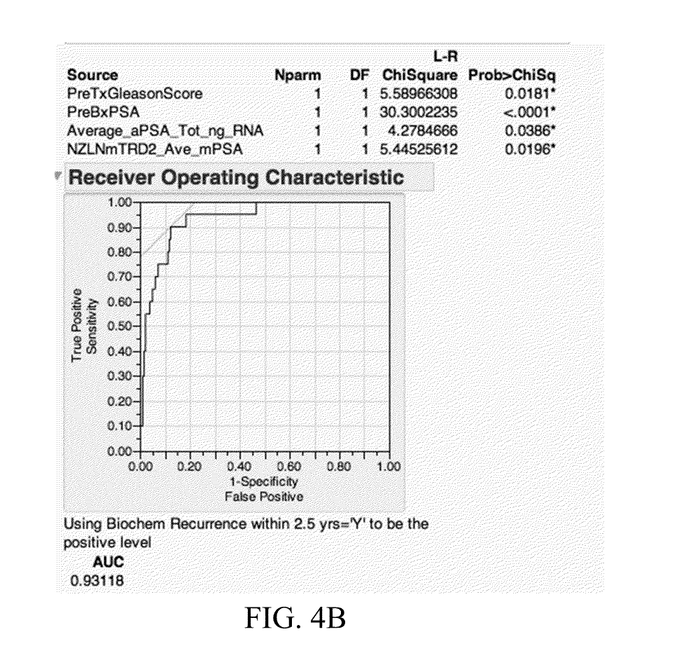

EXAMPLE 6 provides a representative result of a statistical analysis with PPA in combination with a molecular biomarker. Clinical data in ROC analysis is discontinuous. In the ROC analysis algorithm, JMP determines the data point on the ROC curve that lies closest to (0,1) on the (1-specificity, sensitivity) axes (FIG. 4). This point is highlighted in both the numerical and the graphical output, and represents a dichotomous cutoff point associated with a probability table for the test. Above the cutoff value the test is likely positive and below the cutoff value the test is likely negative.

The cutoff value is also represented on the graph allowing for the direct determination of the Sensitivity (y-axis value) and 1-Specificity of the test (x-axis value). These values allow computation of the Positive Predictive Value (PPV) and Negative Predictive Value (NPV) using the prevalence of the event in question. In FIG. 4, prevalence of the event in question is defined as the number of patients who experienced a recurrence within 2.5 years/total number of patients. The area under the curve (AUC) value is a general measure of the success of the test. An AUC approaching 1.0 yields the closest approach of the curve cutoff to the (1,0)-point, representing the point at which both sensitivity and specificity are 1 (i.e., the test is always correct). The closer the AUC value is to 1, the better the predictive nature of the test results. The AUC is 0.79 when serum PSA and Gleason score are combined for the multi-variate analysis (Baseline, shown in FIG. 4A). Surprisingly, the AUC is increased all the way to 0.93 when additional biomarkers of PPA and TXNRD2 are included in the multi-variate analysis (FIG. 4B), approaching the value of 1.0. Despite the slightly unconventional combination of PPA, an enzymatic biomarker, with molecular biomarkers, clearly, the multi-variate analysis of PPA along with other biomarkers has significantly increased the predictive power of PPA.

What is claimed:

1. A method for predicting outcome of prostate cancer in a subject diagnosed with prostate cancer, comprising:
a) determining or obtaining a normalized level of prostate specific antigen (PSA) proteolytic activity (PPA) in a sample from the subject, wherein the normalized level is normalized with a normalization factor;
b) determining or obtaining a gene expression level of one or more molecular biomarkers in the sample or another sample from the subject;
c) performing a multi-variate analysis with the normalized level of PPA and gene expression level of one or more molecular biomarkers and, optionally, one or more additional parameters of the subject; and
d) comparing result(s) of the multi-variate analysis to a cutoff value to predict the outcome of prostate cancer in the subject.

2. The method of claim 1, wherein the step of determining a normalized level of PPA comprises contacting the sample with a labeled prostate cancer-specific peptide that is a substrate of PSA and normalizing the level of PPA with a normalization factor.

3. The method of claim 1 or 2, further comprising normalizing the gene expression level of one or more molecular biomarkers to a normalization factor.

4. The method of claim 3, wherein the normalization factors for normalizing the level of PPA and normalizing the gene expression level(s) are the same.

5. The method of any one of the preceding claims, wherein the sample and/or other sample is a/an expressed prostate secretion (EPS), prostatic fluid (PF), semen, urine, or post prostatic massage urine (PMU) sample.

6. The method of any one of the preceding claims, wherein the normalization factor is serum PSA (sPSA), log of the serum PSA (sPSA), EPS sample volume, log of the EPS sample volume, PSA RNA copy number, log of PSA RNA copy number, amount of RNA, or log of amount of RNA.

7. The method of any one of the preceding claims, wherein the one or more molecular biomarkers comprises any one or more of TXNRD2, TXNRD1, GAPDH, PSA, TMPRSS2:ERG, PCA3 and GAPDH.

8. The method of claim 7, wherein the one or more molecular biomarkers comprises TXNRD2 or TMPRSS2:ERG or both TXNRD2 and TMPRSS2:ERG.

9. The method of any one of the preceding claims, wherein the one or more additional parameters of the subject comprises one or more of age, Gleason score, and level of sPSA.

10. The method of any one of claims 1-6, wherein the multi-variate analysis is performed with the normalized level of PPA, the level of gene expression of one or more molecular biomarkers, or normalized level(s) thereof, and the additional parameters listed in any one of Tables 1-4.

11. The method of any one of the preceding claims, wherein the method further comprises:
i) creating a ROC curve based on one or more values from the multi-variate analysis;
ii) identifying the point on the ROC curve closest to the coordinates (0,1); and
iii) using the closest point to establish the cutoff value.

12. The method of any one of the preceding claims, wherein when the result of the multi-variate analysis is less than the cutoff value, poorer prognosis of prostate cancer is indicated, and when the result is greater than the cutoff value, better prognosis of prostate cancer is indicated.

13. The method of any one of the preceding claims, further comprising a step of treating the subject or providing information regarding a treatment to the subject based on information obtained from the comparison.

14. The method of any one of the preceding claims, wherein the treatment comprises further monitoring, or the method further comprises recommending further monitoring of the subject.

15. The method of any one of the preceding claims, wherein the method further comprises repeating steps a) through d) one or more additional times on one or more samples from the subject to monitor the prostate cancer in the subject.

16. A method of determining a set of parameters to predict the outcome of prostate cancer in a subject diagnosed with prostate cancer, comprising:
a) determining or obtaining a normalized level of PPA in a sample from a subject, wherein the normalized level is normalized with a normalization factor;
b) determining or obtaining a gene expression level of one or more molecular biomarkers in the sample or another sample from the subject;
c) performing one or more multi-variate analyses with the normalized level of PPA and gene expression level of one or more molecular biomarkers and, optionally, one or more additional parameters of the subject; and
d) determining the predictive power of the one or more multi-variate analyses.

17. The method of claim 16, wherein the predictive power is determined by comparing the results of the one or more multi-variate analyses to one or more baseline values.

18. The method of claim 17, wherein the one or more baseline values are one or more cutoff values.

19. The method of claim 16, wherein the predictive power is determined by calculating the area under the curve (AUC).

20. The method of any one of claims 16-19, wherein the step of determining a normalized level of PPA comprises contacting the sample with a labeled prostate cancer-specific peptide that is a substrate of PSA and normalizing the level of PPA with a normalization factor.

21. The method of any one of claims 16-20, further comprising normalizing the gene expression level of one or more molecular biomarkers to a normalization factor.

22. The method of claim 21, wherein the normalization factors for normalizing the level of PPA and normalizing the gene expression level(s) are the same.

23. The method of any one of claims 16-22, wherein the sample and/or other sample is a/an expressed prostate secretion (EPS), prostatic fluid (PF), semen, urine, or post prostatic massage urine (PMU) sample.

24. The method of any one of claims 16-23, wherein the normalization factor is serum PSA (sPSA), log of the serum PSA (sPSA), EPS sample volume, log of the EPS sample volume, PSA RNA copy number, log of PSA RNA copy number, amount of RNA, or log of amount of RNA.

25. The method of any one of claims 16-24, wherein the one or more molecular biomarkers comprises any one or more of TXNRD2, TXNRD1, GAPDH, PSA, TMPRSS2:ERG, PCA3 and GAPDH.

26. The method of claim 25, wherein the one or more molecular biomarkers comprises TXNRD2 or TMPRSS2:ERG or both TXNRD2 and TMPRSS2:ERG.

27. The method of any one of claims 16-26, wherein the one or more additional parameters of the subject comprise one or more of age, Gleason score, and level of sPSA.

28. The method of any one of claims 16-27, wherein the one or more multi-variate analyses is performed with the normalized level of PPA, the level of gene expression of one or more molecular biomarkers, or normalized level(s) thereof, and the additional parameters listed in any one of Tables 1-4.

29. The method of any one of claims 16-28, wherein the method further comprises:
i) creating one or more ROC curves based on one or more values from the one or more multi-variate analyses;
ii) identifying the point(s) on the one or more ROC curves closest to the coordinates (0,1); and
iii) using the closest point(s) to establish one or more cutoff values.
